(19) United States
(12) Patent Application Publication
(10) Pub. No.: US 2008/0138291 A1
(43) Pub. Date: Jun. 12, 2008

(54) Sulfonamide Derivatives Having Carbonic Anhydrase Inhibiting Activity and their Use as Therapeutic and Diagnostic Agents

(75) Inventors: Claudiu T. Supuran, Firenze (IT); Andrea Scozzafava, Florence (IT)

Correspondence Address: YOUNG & THOMPSON, 209 Madison Street, Suite 500, ALEXANDRIA, VA, 22314, US

(73) Assignee: UNION LIFE SCIENCES LTD., Oxford (GB)

(21) Appl. No.: 11/993,468

(22) PCT Filed: Jun. 20, 2006
(86) PCT No.: PCT/IB06/51976

§ 371 Date: Feb. 12, 2008

(30) Foreign Application Priority Data
Jun. 23, 2005 (IT) PCT/IT2005/000366

(51) Int. Cl. A61K 49/00; C07D 311/82; C07D 285/08; A61P 35/00; A61K 31/433; A61K 31/352

(52) U.S. Cl. 424/9.6; 549/392; 548/130; 514/361; 514/454

(57) ABSTRACT

The present invention discloses sulfonamide A-(Q)n—Ar—SChNHR which are CA IX-selective inhibitors, which selectively bind to the enzyme under hypoxic conditions and are able to reverse the tumor acidification mediated by the enzyme. These compounds are useful in anticancer therapies based on tumor-associated CA isozyme inhibition as well as for hypoxic tumor imaging.

The present invention relates to the medical and pharmaceutical field, in particular to sulfonamide derivatives, processes for their preparation, their use as medicaments and diagnostic tools and compositions containing them.

### BACKGROUND OF THE INVENTION

It was known for several years that many sulfonamides possessing carbonic anhydrase (CA, EC 4.2.1.1) inhibitory properties (Supuran, C. T.; et al.; Med. Res. Rev. 2003, 23, 146-189; Supuran, C. T.; Scozzafava, A.; Conway, J. (Eds.) Carbonic anhydrase—its inhibitors and activators, CRC Press (Taylor and Francis Group), Boca Raton, Fla., 2004, pp. 1-363, and references cited therein; Casini, A.; et al.; Curr. Cancer Drug Targets 2002, 2, 55-75; Pastorekova, S.; et al; J. Enz. Inhib. Med. Chem. 2004, 19, 199-229; Scozzafava. A.; et al.; Curr. Med. Chem. 2003, 10, 925-953) also inhibit in various degrees the growth of tumor cells in vitro and in vivo (see above and also Parkkila, S.; Proc. Natl. Acad. Sci. USA 2000, 97, 2220-2224; Teicher, B. A., et al.; Anticancer Res. 1993, 13, 1549-1556, Supuran, C. T.; Scozzafava, A.; Eur. J. Med. Chem. 2000, 35, 867-874; Supuran, C. T.; Scozzafava, A.; J. Enz. Inhib. 2000, 15, 597-610; Scozzafava, A.; Supuran, C. T.; Bioorg. Med. Chem. Lett. 2000, 10, 1117-1120; C. T. Supuran, et al; Bioorg. Med. Chem. 2001, 9, 703-714). The precise isozyme(s) involved in such processes, among the 15 presently characterized human CAs, were not known up till recently, but the discovery of CA IX (Pastorek, J., et al; Oncogene 1994, 9, 2788-2888; Opavský, R.; et al; Genomics 1996, 33, 480-487) and then of CA XII (Tureci, O.; et al; Proc. Natl. Acad. Sci. USA 1998, 95, 7608-7613) as isozymes predominantly present in tumors, offered a starting point for more detailed studies in the field. Another issue little understood in the first years of “CA—tumors connection” research was why various tumor cell lines belonging to the same tumor type (for example leukemia, non-small cell lung cancer, ovarian, melanoma, colon, CNS, renal, prostate or breast cancer) showed very different sensitivity to inhibition by sulfonamides, with GI₅₀ (molarity of inhibitor producing a 50% inhibition of tumor cell growth) values typically in the range of 30 μM-10 nM (Eur. J. Med. Chem. 2000, 35, 867-874; J. Enz. Inhib. 2000, 15, 597-610; Bioorg. Med. Chem. Lett. 2000, 10, 1117-1120; Bioorg. Med. Chem. 2001, 9, 703-714). It has been discovered only later that CA IX/XII are not present in all tumor types, (Carbonic anhydrase—its inhibitors and activators, CRC Press (Taylor and Francis Group), Boca Raton, Fla., 2004, pp. 1-363, and references cited therein; J. Enz. Inhib. Med. Chem. 2004, 19, 199-229; Curr. Med. Chem. 2003, 10, 925-953) and furthermore, that the levels of isozyme IX—the best studied one at this moment—dramatically increase in response to hypoxia via a direct transcriptional activation of the CA9 gene by the hypoxia inducible factor HIF-1 (Wykoff, C. C.; et al; Cancer Res. 2000, 60, 7075-7083). It has also been proven thereafter that the expression of CA IX in tumors is a sign of poor prognosis (Potter, C. P. S.; Harris, A. L.; Brit. J. Cancer 2003, 89, 2-7).

Acidic extracellular pH (pHe) has been associated with tumor progression via multiple mechanisms including up-regulation of angiogenic factors, proteases, increased invasion, and impaired immune functions (Stubbs, M.; et al; Mol. Med. Today 2000, 6, 15-19; Helmlinger, G.; et al; Clin. Cancer Res. 2002, 8, 1284-1291; Fukumura, D.; et al; Cancer Res. 2001, 61, 6020-6024; Kato, Y.; et al; J. Biol. Chem. 1992, 267, 11424-11430; Martinez-Zaguilan, et al; Clin. Exp. Metastasis 1992, 14, 176-186; Fischer, B.; et al; Clinical Immunol. 2000, 96, 252-263).

In addition, acidic pHe can influence uptake of anticancer drugs and modulate response of tumor cells to conventional chemo- and radiotherapy (Carbonic anhydrase—its inhibitors and activators, CRC Press (Taylor and Francis Group), Boca Raton, Fla., 2004, pp. 1-363, and references cited therein; J. Enz. Inhib. Med. Chem. 2004, 19, 199-229; Curr. Med. Chem. 2003, 10, 925-953). Acidification of the tumor microenvironment was generally assigned to be due to accumulation of lactic acid excessively produced by glycolysis and poorly removed by inadequate tumor vasculature (Mol. Med. Today 2000, 6, 15-19; Clin. Cancer Res. 2002, 8, 1284-1291; Newell, K; et al.; Proc. Natl. Acad. Sci. USA 1993, 90, 1127-1131). The high rates of glycolysis are important for hypoxic cells which largely depend on anaerobic metabolism for their energy generation (Mol. Med. Today 2000, 6, 15-19; Clin. Cancer Res. 2002, 8, 1284-1291; Newell, K; et al.; Proc. Natl. Acad. Sci. USA 1993, 90, 1127-1131). However, experiments with glycolysis-deficient cells recently indicated that production of lactic acid is not the only mechanism leading to tumor acidification. Glycolysis-deficient cells were shown to produce only diminished amounts of lactic acid, but form acidic tumors anyhow in vivo (Proc. Natl. Acad. Sci. USA 1993, 90, 1127-1131). Comparison of the metabolic profiles of the glycolysis-impaired and parental cells revealed that another molecule—CO₂—in addition to lactic acid, is a significant source of acidity in tumors (Mol. Med. Today 2000, 6, 15-19; Clin. Cancer Res. 2002, 8, 1284-1291). Since CO₂ hydration is a very slow process without catalysts at the physiological pH, the presence of enzymes involved in the interconversion between carbon dioxide and bicarbonate is essential for the housekeeping cell necessities, and these enzymes are the CAs. Based on several distinctive properties, the tumor associated isozyme CA IX appeared to be the best candidate for a role in acidification of the tumor microenvironment. Thus, CA IX is an integral plasma membrane protein with an extracellularly exposed enzyme active site, (Carbonic anhydrase—Its inhibitors and activators, Supuran, C. T., Scozzafava, A., Conway, J.; Eds., CRC Press, Boca Raton (Fla.), USA, 2004, pp. 253-280; Bioorg. Med. Chem. 2001, 9, 703-714) possesses a high catalytic activity (Carbonic anhydrase—Its inhibitors and activators, Supuran, C. T., Scozzafava, A., Conway, J.; Eds., CRC Press, Boca Raton (Fla.), USA, 2004) and is present only in few normal tissues, but its ectopic expression is strongly associated with many types of tumors (Potter, C. P. S.; Harris, A. L.; Brit. J. Cancer 2003, 89, 2-7). Finally, CA IX levels dramatically increase in response to hypoxia via a direct transcriptional activation of the CA9 gene by HIF-1, (Wyhoff, C. C.; et al; Cancer Res. 2000, 60, 7075-7083; Fischer, B.; Clinical Immunol. 2000, 96, 252-263). Thus, CA IX has all the necessary requisites to act in tumor pH control.

Brubaker, K.; et al. (The Journal of Histochemistry and Cytochemistry, Vol. 47(4): 545-550; 1999) describes Bodipy 558/568-modified acetazolamide for localization of Carbonic Anhydrase in osteoclasts, CA II and IV being the most sensitive forms.

Sulfonamide derivatives having specific Carbonic Anhydrase IX inhibiting activity are described in WO 2004/048544.

Contrarily to the belief in the state of the art, the present inventors have discovered the capacity of CA IX, and not of lactic acid, to acidify the extracellular pH under hypoxic conditions.

Svastová, E.; et al. (FEBS Letters, 19 Nov. 2004, Vol. 577, no. 3, 439-445) disclose (4-sulfamoylphenylmethyl)thioureido fluorescein as selective inhibiting agent of CA IX. This compound is believed to encounter cytoplasmic accumulation, due to hypoxia-induced CA IX internalization (Svastová, E.; et al.; Exp. Cell. Res., 2003, 290, 332-345).

Keeping CA IX inhibiting agents outside the cell is important in order to prevent their possible activity on different CAs inside the cell, thus giving rise to side effects.

This problem has been faced in the above mentioned WO 2004/048544 and solved with sulfonamide derivatives bearing a pyridilum residue. The cationic moiety makes the compound membrane impermeant, see also the above mentioned Svastová, E.; et al.; FEBS Letters, 19 Nov. 2004, Vol. 577, no. 3, 439-445.

As explained above, low extracellular pH is a critical factor in tumor progression and treatment, therefore, there is a need to control and even better to reverse extracellular pH in tumor environment.

It has now been found that certain sulfonamides bearing a fluorescent moiety possess potent CA IX inhibitory properties and are able to reverse extracellular pH occurring under hypoxic conditions.

### SUMMARY OF THE INVENTION

It is an object of the present invention a novel class of strong CA IX inhibitors bearing fluorescent tails, which are useful for imaging this isozyme in hypoxic tumors or for inhibiting it, with restoration of the normal pH.

Object of the present invention are compounds of formula (I)

A-(Q)ₙ-Ar—SO₂NHR

wherein
A is the moiety of a fluorescent dye;
Q is the group —NH—CX—NH—(R₁)ₘ or —NH—CX—NH—NH—(R₁)ₘ,
   wherein
X is O or S, R₁ is a C₁-C₄ alkylene, m is the number 0 or 1;
n is the number 0 or 1;
Ar is a C₆-C₁₀ aromatic or a heteroaromatic group containing at least one heteroatom selected from the group consisting of oxygen, nitrogen and sulphur, said aromatic and heteroaromatic groups optionally being substituted by at least one, halogen atom;
R is hydrogen or a B—SO₂NH₂ group, wherein B is a (C₁-C₄)ᵣ alkylene-aromatic or (C₁-C₄)ᵣ alkylene-heteroaromatic group, wherein r is 0 or 1;
with the exclusion of the (4-sulfamoylphenylmethyl)thioureido fluorescein,
their pharmaceutically acceptable hydrates, solvates and salts.

The compounds according to the present invention show the unexpected property of a selective inhibiting activity of tumor-related Carbonic Anhydrase IX with respect to ubiquitary Carbonic Anhidrases I and II.

Moreover, the compounds according to the present invention do not pass cell membrane, thus enhancing the selective activity.

Another important characteristic of the compounds of the present invention is their ability to reverse acidic extracellular pH in hypoxic tumors.

These compounds are therefore useful as diagnostic and therapeutic agents as it will be disclosed in detail in the following sections of the description.

Further objects of the present invention are processes for the preparation of the compounds of formula (I), their use for the preparation of medicaments and diagnostic tools, in particular in the field of tumors, as well as methods for the diagnosis and treatment of tumors.

Other objects of the present invention are compositions comprising the compounds of formula (I), in particular pharmaceutical and diagnostic compositions.

These and other objects of the present invention will be disclosed in further detail also by means or Examples and Figures.

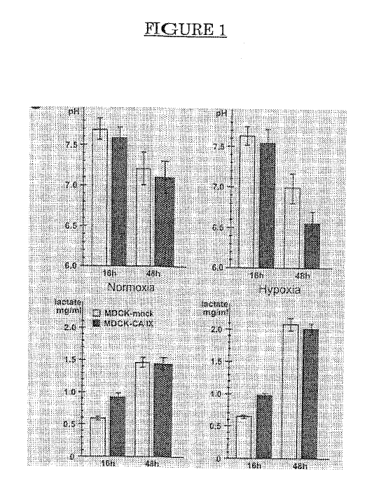

FIG. 1 shows the values of pHe and lactate concentrations in CA IX-transfected MDCK cells and mock-transfected controls in hypoxia (H, 2% O₂)/normoxia (N, 21% O₂).

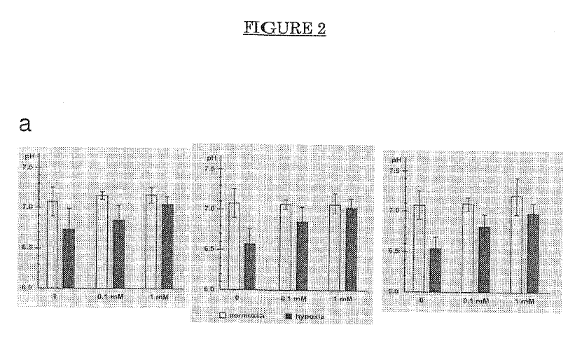

FIG. 2 shows the binding of three different sulfonamide CAIs (including the fluorescent derivative 5c according to the invention) to hypoxic MDCK-CA IX cells and their effect on the pHe.

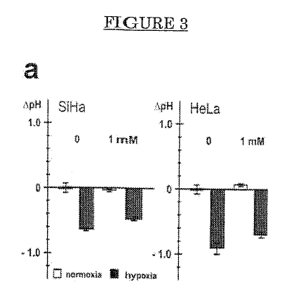

FIG. 3 shows treatment of the tumor HeLa and SiHa cervical carcinoma cells with the fluorescent sulfonamide 5c and its effect on the tumor pH.

### DETAILED DESCRIPTION OF THE INVENTION

According to the present invention, the group A in formula (I) represents the moiety of a fluorescent dye. The term is well understood by the person of ordinary skill in the art. Examples of definitions are given in U.S. Pat. No. 5,919,922 and the references cited therein and all available commercial catalogues. A preferred example of fluorescent dye is fluorescein (CAS RN 2321-07-05).

The group Q is the group —NH—CX—NH—(R₁)ₘ, wherein X is O or S, R₁ is a C₁-C₄ alkylene, m is the number 0 or 1.

C₆-C₁₀ aromatic group means phenyl or 1- or 2-naphthyl.

Heteroaromatic group means a C₃-C₁₂ carbocyclic compound containing at least one heteroatom selected from the group consisting of oxygen, nitrogen and sulphur. 1,3,4-thiadiazole is a preferred heterocyclic group.

Preferred C₁-C₄ alkylene groups are methylene, ethylene. Alkylene groups can also be branched, but the total number of carbon atoms is maximum 4.

Fluorine, chlorine, iodine and bromine are preferred halogen atoms.

Pharmaceutically acceptable salts and solvates are well known to the person of ordinary skill in the art and need no further explanation. See for example Wermuth, C. G. and Stahl, P. H. (eds.) Handbook of Pharmaceutical Salts, Properties; Selection and Use; Verlag Helvetica Chimica Acta, Zürich, 2002. Examples of suitable salts are sodium, potassium, litium, amines.

A first preferred group of compounds of formula (I) are those wherein Q is the group —NH—CX—NH-(i)ₘ, wherein X is O or S, R₁ is a C₁-C₄ alkylene, m is the number 0 or 1, Ar is phenyl, optionally substituted by at least one halogen atom and R is H.

A second preferred group of compounds of formula (I) are those wherein Q is the group —NH—CX—NH—(R₁)ₘ, wherein X is S, m is 0, Ar is phenyl, optionally substituted by at least one halogen and R is H.

A third preferred group of compounds of formula (I) are those wherein Q is the group —NH—CX—NH—(R₁)ₘ, wherein X is S, R₁ is a C₁-C₂ alkylene, m is 1, Ar is phenyl, optionally substituted by at least one halogen and R is H.

A fourth preferred group of compounds of formula (I) are those wherein Q is the group —NH—CX—NH—(R₁)ₘ, wherein X is S, m is 0, Ar is phenyl and R is B—SO₂NH₂ group, wherein B is 1,3,4-thiadiazol-2-yl.

In all the groups of preferred compounds, the most preferred fluorescent dye residue A is fluorescein.

There is no limitation as to the possible position of the groups. For example, any possible position of the fluorescent dye moiety A can bear the remainder of the molecule, as well as any possible position of the group Ar can bear the group SO₂—NHR and A-(Q)ₙ, respectively. The same applies when B is an aromatic or heteroaromatic group.

According to the present invention, particularly preferred compounds are:
(4-Sulfamoylphenyl)thioureido fluorescein;
(4-Sulfamoylphenylethyl)thioureido fluorescein;
(2-Iodo-4-sulfamoylphenyl)thioureido fluorescein;
(3-Sulfamoylphenyl)thioureido fluorescein;
[4-(4-Sulfamoyl-benzylsulfamoyl)-phenyl]-thioureido fluorescein;
[4-(5-Sulfamoyl-[1,3,4]thiadiazol-2-ylsulfamoyl)-phenyl]-thioureido fluorescein.

The preferred compounds are shown in the following scheme.

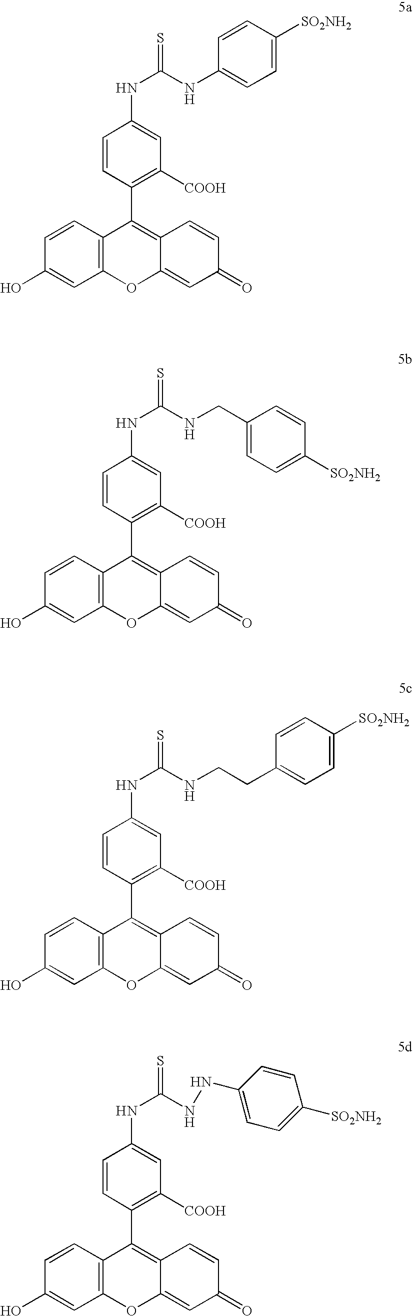

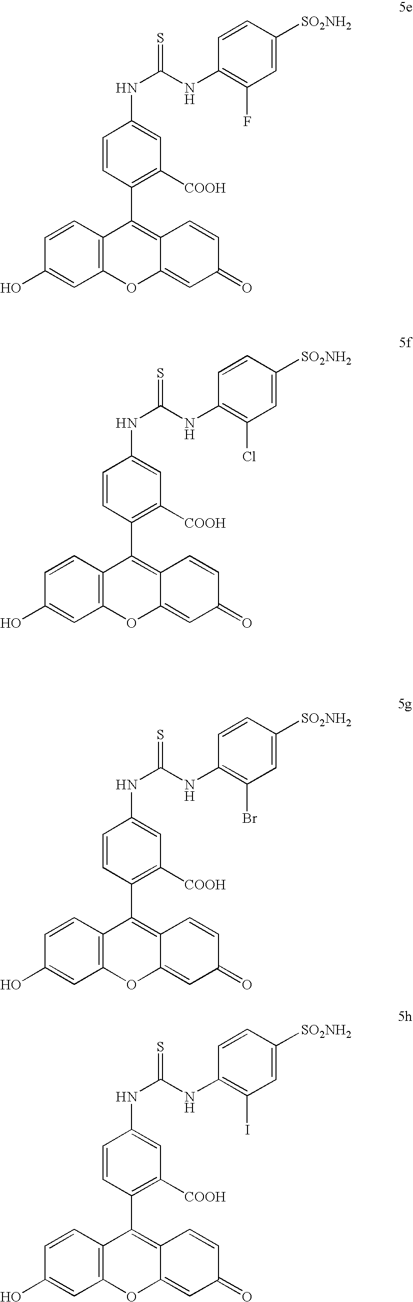

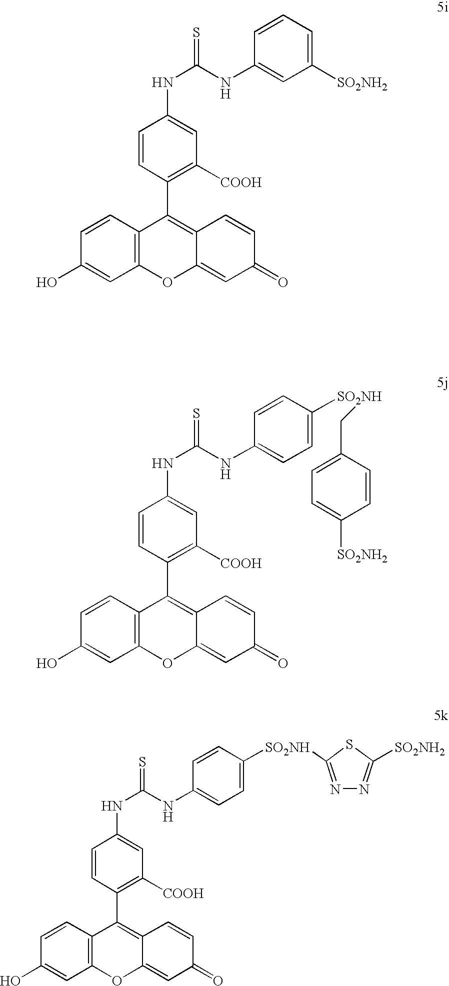

Another group of preferred compounds is the following:
(4-Sulfamoylphenyl)ureido fluorescein (1);
(3-Sulfamoylphenyl)ureido fluorescein (2);
(9-Sulfamoylphenyl)ureido fluorescein (3);
(4-Sulfamoylphenylmethyl)ureido fluorescein (4);
(4-Sulfamoylphenylethyl)ureido fluorescein (5);
(2-Fluoro-4-sulfamoylphenyl)ureido fluorescein (6);
(2-Chloro-4-sulfamoylphenyl)ureido fluorescein (7);
(2-Bromo-4-sulfamoylphenyl)ureido fluorescein (8);
(2-Iodo-4-sulfamoylphenyl)ureido fluorescein (9);
[4-(4-Sulfamoyl-benzylsulfamoyl)-phenyl]-ureido fluorescein (10);
[4-(5-Sulfamoyl-[1,3,4]thiadiazol-2-ylsulfamoyl)-phenyl]-ureido fluorescein (11).

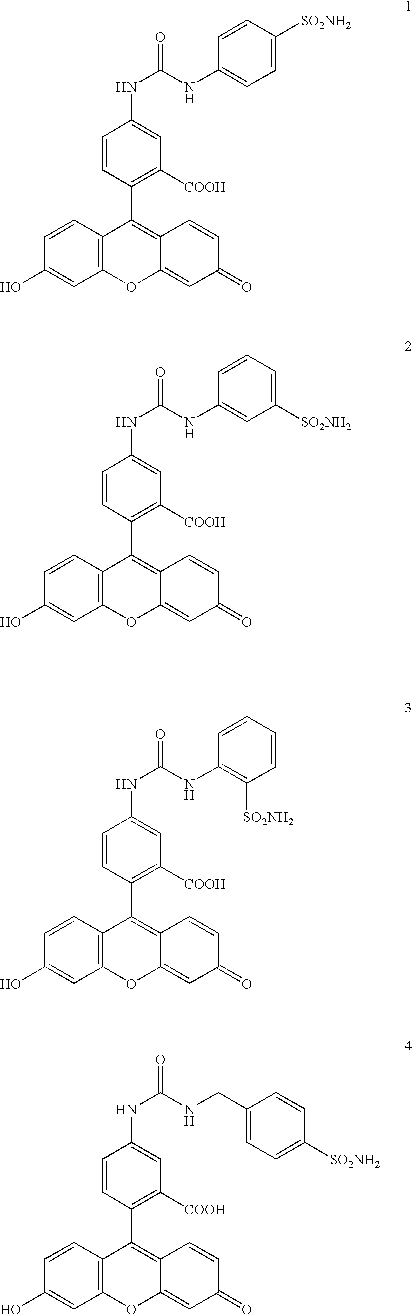

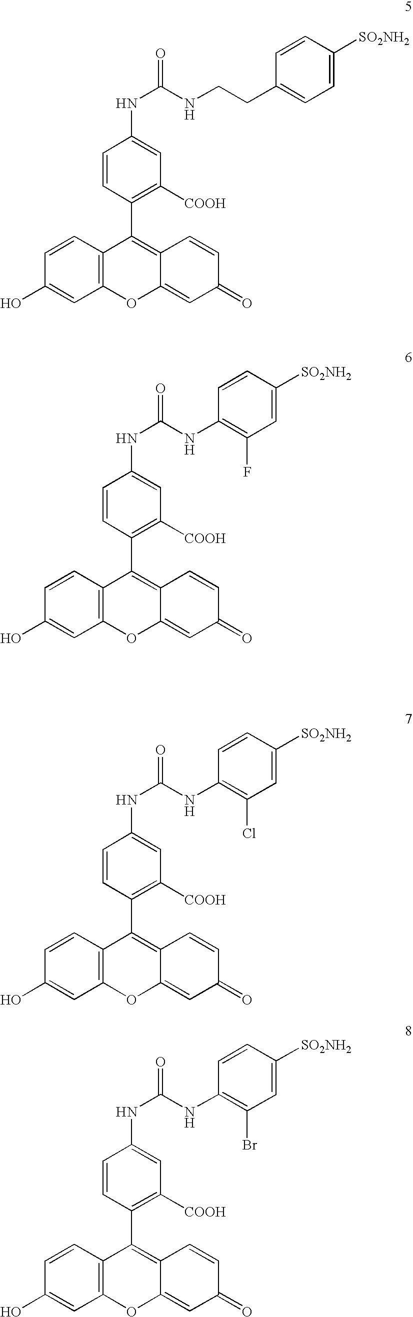

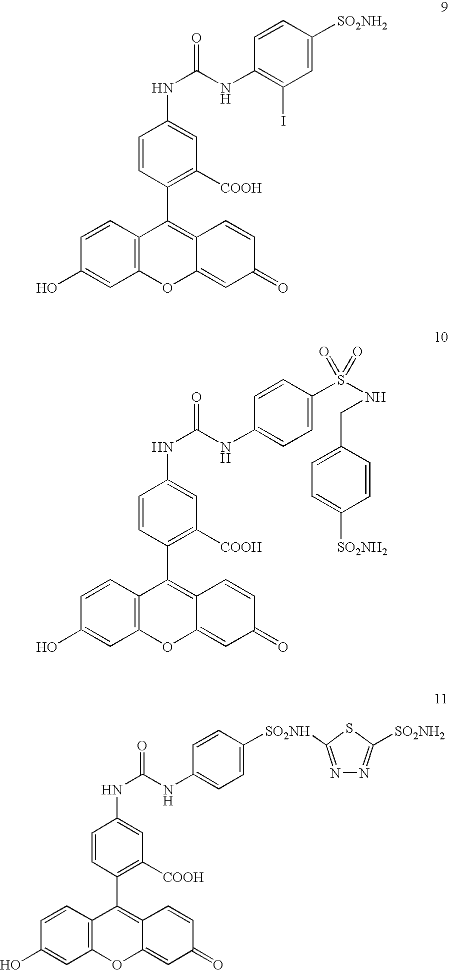

Another object of the present invention is a process for the preparation of the compounds of formula (I), wherein A is a as defined above, preferably a fluorescein residue, comprising the reaction of a compound of formula (II) A-NH₂, wherein A is as defined above, with a compound of formula (III) XC—NH—(R₁)ₘ—Ar—SO₂NHR, wherein X, R₁, m and Ar are as defined above.

Alternatively, the reaction of is carried out between a compound of formula (IV) A-NCX, wherein A and X are as defined above, with a compound of formula (V) H₂N—(R₁)ₘ—Ar—SO₂NHR, wherein R₁, m and Ar are as defined above.

Reaction conditions are those known to the ones skilled in the art and does not need any particular description, see for example, Casini, A.; et al.; J. Med. Chem., 2000, 43, 4884-4892; Innocenti, A.; et al.; J. Med. Chem., 2004, 47, 5224-5229.

In a second embodiment according to the present invention, the compounds of formula (I) can be prepared by reaction of fluorescein isothiocyanate (FITC) 2 with amino/hydrazino-substituted aromatic/heterocyclic sulfonamides 4, as previously reported for structurally related thioureas (Supuran, C. T.; et al.; Eur. J. Med. Chem. 1998, 33, 83-93).

The following Scheme 1 shows an exemplary embodiment of the process according to the present invention.

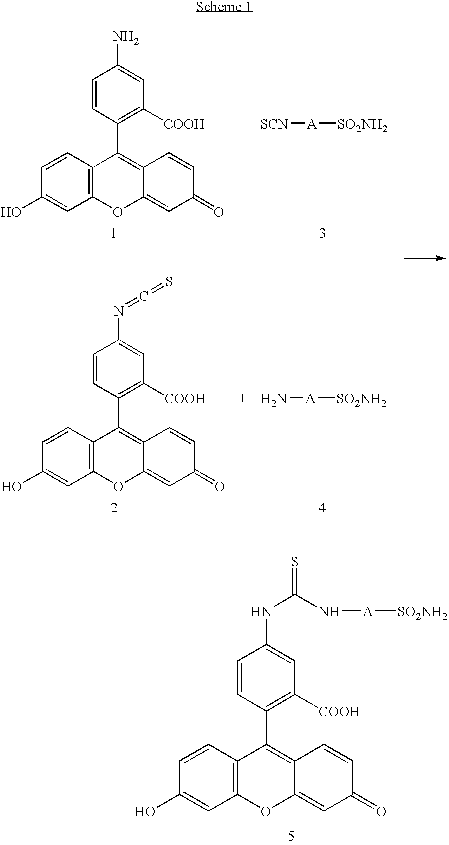

Generally, in the first process, fluorescein amine (1) and the isothiocyanate sulfonamide (3), preferably in equimolar amounts, are dissolved in a suitable organic solvent, such as for example N,N-dimethylacetamide or equivalent, and the resulting mixture is stirred at a temperature which does not affect the reaction, for example room temperature. The reaction is left to proceed until completion (monitoring), and subsequently is dissolved in water and extracted with a suitable solvent (for example ethylacetate). The desired product is present in the organic layer, which is usually dried (for example over anhydrous sodium sulphate), filtered and concentrated under vacuum. If desired, the resulting product is then purified by usual techniques, such as for example flash chromatography.

In the second process, fluorescein isothiocyanate and the amino sulfonamide derivative preferably in equimolar amounts, are dissolved in a suitable organic solvent, such as for example N,N-dimethylacetamide or equivalent, then a sufficient amount of organic amine, such as triethylamine, for example in equimolar amount is added and the resulting mixture is stirred at a temperature which does not affect the reaction, for example room temperature. The reaction is left to proceed until completion (monitoring), and subsequently is dissolved in water and extracted with a suitable solvent (for example ethylacetate). The desired product is present in the organic layer, which is usually dried (for example over anhydrous sodium sulphate), filtered and concentrated under vacuum. If desired, the resulting product is then purified by usual techniques, such as for example flash chromatography.

The ureido-fluoresceinyl sulfonamides are prepared by condensing amino fluorescein A (commercially available derivative) with isocyanato-sulfonamides B (prepared from the corresponding aminosulfonamides, most of which are commercially available derivative) and phosgene (H₂N—R—SO₂NH₂+COCl₂═OCN—R—SO₂NH₂+2 HCl), in refluxing toluene, as described in the literature (Smith, J.; J. Org. Chem. 1965, 30, 1260-1262) (Scheme 2). The ureido-compounds are in fact synthesized similarly to the corresponding thioureido ones, described above.

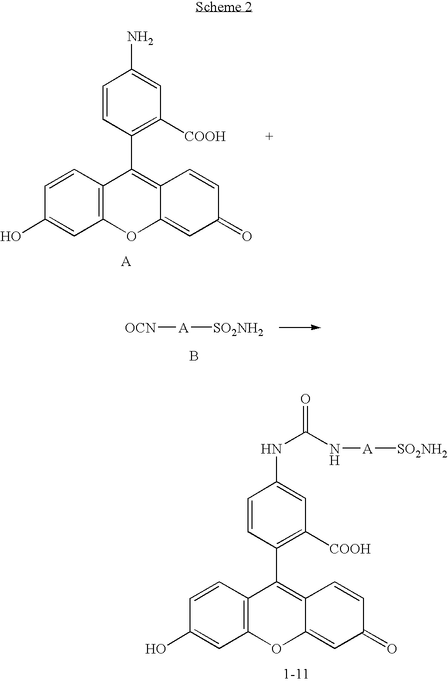

The compounds according to the present invention are selective Carbonic Anhydrase IX inhibitors.

Due to this property, they are useful as probes for the identification of hypoxic tumors. In particular, the tumor to be identified is a Carbonic Anhydrase IX-positive tumor.

The identification of tumor is intended in its broadest meaning. Identification can be carried out either in vivo, namely on a living body, or in vitro, i.e. on a sample of tumor tissue taken from a subject affected or suspect to be affected by such a tumor. Any method using fluorescent detection is suitable. A preferred method is positron-emission tomography.

In the embodiment providing the in vivo use of the probe, the compound is intended useful for the preparation of a reagent for the detection of Carbonic Anhydrase, in particular for the detection of Carbonic Anhydrase IX, more in particular for the detection of Carbonic Anhydrase IX-positive tumors.

Another object of the present invention is a fluorescent reagent comprising a compound of formula (I). This reagent is useful for the detection of tumor cells expressing membrane bound Carbonic Anhydrase IX. The reagent can be incorporated in a composition part of a diagnostic kit for tumor imaging. The conventional preparation of said fluorescent reagent and the related composition and kit are well known in the art (see for example WO 98/41649 and references cited therein).

The compounds of formula (I) are useful for the preparation of a medicament.

In a preferred embodiment of the present invention, the medicament has carbonic anhydrase inhibiting action, more preferably toward carbonic anhydrase isozyme IX.

Thanks to these properties, the compounds of formula (I) are particularly useful in a medicament or in a method for the treatment of a hypoxic tumor. Examples of this kind of tumors are kidney, breast, lung, head and neck, gliomas, mesotheliomas, stomach, colon, biliary, pancreatic, cervix, endometrial, squamal/basal cell carcinomas.

More particularly, the medicament is effective for reversing acidification of a hypoxic tumor.

The medicament according to the present invention is effective for treating a Carbonic Anhydrase IX-positive tumor.

The said medicament of the present invention can be used in combination therapy, for example antitumor therapy. Antitumor therapy is intended in its broadest sense, including chemotherapy, radiotherapy, combined therapy.

In accordance with the present invention, the pharmaceutical compositions contain at least one active ingredient in an amount such as to produce a significant therapeutic effect. The compositions covered by the present invention are entirely conventional and are obtained with methods that are common practice in the pharma-ceutical industry, such as, for example, those illustrated in Remington's Pharmaceutical Science Handbook, Mack Pub. N.Y.—latest edition. According to the administration route opted for, the compositions will be in solid or liquid form, suitable for oral, parenteral or intravenous administration. The compositions according to the present invention contain, along with the active ingredient, at least one pharmaceutically acceptable vehicle or excipient. Formulation adjuvants may be particularly useful, e.g. solubilising agents, dispersing agents, suspension agents or emulsifying agents.

The following examples further illustrate the invention.

General: ¹H-NMR spectra were recorded on a Bruker DRX-400 spectrometer using DMSO-d₆ as solvent and tetramethylsilane as internal standard. Chemical shifts are expressed in δ (ppm) downfield from tetramethylsilane, and coupling constants (J) are expressed in Hertz. Electron Ionization mass spectra (30 eV) were recorded in positive or negative mode on a Water MicroMass ZQ.

### EXAMPLE 1

### General Procedure for the Preparation of Compounds of Formula (I)

### Method A:

Fluorescein isothiocyanate (0.001 mole) and the amino sulfonamide derivative (0.001 mole) were dissolved in 5 ml of dimethylformamide; then triethylamine (0.001 mole) was added and the mixture was stirred at room temperature until completion of the reaction (TLC monitoring). The reaction mixture was then dissolved in water and was extracted with ethylacetate; the organic layer was dried over anhydrous sodium sulfate, filtered and concentrated under vacuum. The resulting product was then purified by flash chromatography.

### Method B:

Fluorescein amine (0.001 mole) and the isothiocyanate sulfonamide (0.001 mole) were dissolved in 5 ml of N,N-dimethylacetamide and then the mixture was stirred at room temperature until completion of the reaction (TLC monitoring). The reaction mixture was then dissolved in water and extracted with ethylacetate; the organic layer was dried over anhydrous sodium sulfate, filtered and concentrated under vacuum. The resulting product was then purified by flash chromatography.

According to the above methods and using the suitable reagents, the following compounds were obtained.

(4-Sulfamoylphenyl)thioureido fluorescein (5a): ¹H NMR (DMSO-d₆, 400 MHz) δ 10.45 (s, 1H), 10.35 (s, 1H), 10.15 (s, 2H), 8.2 (d, 1H, J=1.85 Hz), 7.85 (dd, 1H, J=2 Hz), 7.8 (d, 2H, J=8.7 Hz), 7.7 (d, 2H, J=8.7 Hz), 7.35 (s, 2H), 7.25 (d, 2H, J=8.2 Hz), 6.7 (d, 2H, J=2 Hz), 6.6 (m, 4H); MS ESI⁺ m/z 562 (M+H)⁺. ESI⁻ m/z 560 (M−H)⁻.

(4-Sulfamoylphenylethyl)thioureido fluorescein (5c): ¹H NMR (DMSO-d₆, 400 MHz) ¹H NMR (DMSO-d₆, 400 MHz) δ 10.15 (s, 2H), 9.95 (s, 1H), 8.25 (s, 1H), 8.1 (s, 1H), 7.8 (d, 2H, J=6.6 Hz), 7.7 (d, 1H, J=8.1 Hz), 7.5 (d, 2H, J=8.3 Hz), 7.3 (s, 2H), 7.2 (d, 1H, J=8.3 Hz), 6.7 (d, 2H, J=4.1 Hz), 6.65-6.55 (m, 4H), 3.8 (q, 2H, J=7.3 Hz, J=4.8 Hz), 3 (t, 2H, J=7.4 Hz); MS ESI⁺ m/z 590 (M+H)⁺. ESI⁻ m/z 588 (—H)—.

(2-Iodo-4-sulfamoylphenyl)thioureido fluorescein (5h): ¹H NMR (DMSO-d₆, 400 MHz) δ 10.45 (s, 1H), 10.15 (s, 2H), 9.8 (s, 2H), 8.3 (dd, 2H, J=15.8 Hz, J=1.8 Hz), 7.63 (d, 1H, J=8.3 Hz), 7.5 (s, 2H), 7.26 (d, 1H, J=8.3 Hz), 6.7 (d, 2H, J=2.3 Hz), 6.6 (m, 4H); MS ESI m/z 686 (M−H)⁻.

(3-Sulfamoylphenyl)thioureido fluorescein (51): 1 NMR (DMSO-d₆, 400 MHz) δ 10.4 (s, 1H), 10.35 (s, 1H), 10.15 (s, 2H), 8.2 (d, 1H, J=1.7 Hz), 7.97 (d, 1H, J=1.6 Hz), 7.83 (dd, 1H, J=8.3 Hz, J=1.8 Hz), 7.75 (d, 1H, J=8 Hz), 7.62 (dd, 1H, J=6.61 Hz, J=1.4 Hz), 7.55 (t, 1H, J=7.8 Hz), 7.44 (s, 2H), 7.24 (d, 1H, J=8.3 Hz), 6.7 (d, 2H, J=2.1 Hz), 6.6 (m, 4H); MS ESI⁺ m/z 562 (M+H)⁺, 584 (M+Na)⁺. EST− m/z 560 (M−H)⁻.

[4-(4-(4-Sulfamoyl-benzylsulfamoyl)-phenyl]-thioureido fluorescein (5j) ¹H NMR (DMSO-d₆, 400 MHz) δ 10.45 (2s, 2H), 10.25 (s, 2H), 8.23 (d, 2H, J=6 Hz), 7.8 (m, 7H), 7.48 (d, 2H, J=8.2 Hz), 7.35 (s, 2H), 7.25 (d, 1H, J=8.2 Hz), 6.7 (d, 2H, J=1.7 Hz), 6.6 (m, 4H), 4.1 (d, 2H, J=5.9 Hz); MS ESI⁺ m/z 731 (M+H)⁺, 753 (M+Na)⁺. ESI⁻ m/z 729 (M−H)⁻.

[4-(5-Sulfamoyl-[1,3,4]thiadiazol-2-ylsulfamoyl)-phenyl]-thioureido fluorescein (5k): ¹H NMR (DMSO-d₆, 400 MHz) δ 10.45 (s, 2H), 10.15 (s, 3H), 8.4 (s, 1H), 8.45 (m, 1H), 8.25 (dd, 1H, J=9.8 Hz, J=1.8 Hz), 7.83 (m, 3H), 7.73 (m, 2H), 7.25 (dd, 1H, J=12.4 Hz, J=8.3 Hz), 6.7 (d, 2H, J=2.5 Hz), 6.6 (m, 4H); MS. ESI⁻ m/z 723 (M−H)⁻.

The ureido-fluoresceinyl sulfonamides were prepared by condensing amino fluorescein (commercially available derivative, Sigma-Aldrich, Milan, Italy) with isocyanato-sulfonamides (prepared from the corresponding aminosulfonamides, most of which are commercially available derivative) and phosgene (H₂N—R—SO₂NH₂+COCl₂═OCN—R—SO₂NH₂+2HCl), in refluxing toluene.

According to the above methods and using the suitable reagents, the following compounds were obtained.

(4-Sulfamoylphenyl)ureido fluorescein (1): ¹H NMR (DMSO-d₆, 400 MHz) δ 10.49 (s, 1H), 10.38 (s, 1H), 10.21 (s, 2H), 8.25 (d, 1H, J=1.85 Hz), 7.85 (dd, 1H, J=2 Hz), 7.81 (d, 2H, J=8.7 Hz), 7.72 (d, 2H, J=8.7 Hz), 7.35 (s, 2H), 7.21 (d, 2H, J=8.2 Hz), 6.73 (d, 2H, J=2 Hz), 6.60 (m, 4H); MS ESI⁺ m/z 546 (M+H)⁺. ESI⁻ m/z 544 (M−H)⁻.

(3-Sulfamoylphenyl)ureido fluorescein (2): ¹H NMR (DMSO-d₆, 400 MHz) δ 10.51 (s, 1H), 10.43 (s, 1H), 10.23 (s, 2H), 8.25 (d, 1H, J=1.7 Hz), 7.95 (d, 1H, J=1.6 Hz), 7.83 (dd, 1H, J=8.3 Hz, J=11.8 Hz), 7.78 (d, 1H, J=8 Hz), 7.64 (dd, 1H, J=6.6 Hz, J=1.4 Hz), 7.52 (t, 1H, J=7.8 Hz), 7.46 (s, 2H), 7.27 (d, 1H, J=8.3 Hz), 6.68 (d, 2H, J=2.1 Hz), 6.60 (m, 4H); MS ESI⁺ m/z 546 (M+H)⁺. ESI⁻ m/z 544 (M−H)⁻.

(2-Sulfamoylphenyl)ureido fluorescein (3): ¹H NMR (DMSO-d₆, 400 MHz) δ 10.45 (s, 1H), 10.35 (s, 1H), 10.18 (s, 2H), 8.20 (d, 1H, J=1.85 Hz 7.95 (d, 1H, J=1.6 Hz), 7.83 (dd, 1H, J=8.3 Hz, J=1.8 Hz), 7.78 (d, 1H, J=8 Hz), 7.64 (dd, 1H, J=6.6 Hz, J=1.4 Hz), 7.35 (s, 2H), 7.25 (d, 2H, J=8.2 Hz), 6.7 (d, 2H, J=2 Hz), 6.6 (m, 4H); MS ESI⁺ m/z 546 (M+H)⁺. ESI⁻ m/z 544 (M−H)⁻.

(4-Sulfamoylphenylmethyl)ureido fluorescein (4): ¹H NMR (DMSO-d₆, 400 MHz) δ 10.62 (s, 1H), 9.13 (s, 1H), 8.24 (s, 1H), 7.83 (d, 2H, J=8.1 Hz), 7.75 (d, 1H, J=8.0 Hz), 7.56 (d, 2H, J=8.1 Hz), 7.17 (d, 1H, J=8.0 Hz), 6.70-6.5 (m, 6H); MS ESI⁺ m/z 560 (M+H)⁺. ESI⁻ m/z 558 (M−H)⁻.

(4-Sulfamoylphenylethyl)ureido fluorescein (5): ¹H NMR (DMSO-d₆, 400 MHz): δ 10.63 (s, 1H), 9.34 (s, 1H), 8.26 (s, 1H), 7.81 (d, 2H, J=8.1 Hz), 7.78 (d, 1H, J=8.0 Hz), 7.54 (d, 2H, J=8.1 Hz), 7.17 (d, 1H, J=8.0 Hz), 6.70-6.5 (m, 6H), 3.13 (t, 2H); MS ESI⁺ m/z 574 (M+H)⁺. ESI⁻ m/z 572 (M−H)⁻.

(2-Fluoro-4-sulfamoylphenyl)ureido fluorescein (6): ¹H NMR (DMSO-d₆, 400 MHz) δ 10.40 (s, 1H), 10.21 (s, 2H), 9.88 (s, 2H), 8.38 (dd, 2H, J=15.8 Hz, J=1.8 Hz), 7.61 (d, 1H, J=8.3 Hz), 7.47 (s, 2H), 7.29 (d, 1H, J=8.3 Hz), 6.70 (d, 2H, J=2.3 Hz), 6.62 (m, 4H); MS ESI⁻ m/z 562 (M−H)⁻.

(2-Chloro-4-sulfamoylphenyl)ureido fluorescein (7): ¹H NMR (DMSO-d₆, 400 MHz) δ 10.46 (s, 1H), 10.17 (s, 2H), 9.81 (s, 2H), 8.36 (dd, 2H, J=15.8 Hz, J=1.8 Hz), 7.60 (d, 1H, J=8.3 Hz), 7.45 (s, 2H), 7.28 (d, 1H, J=8.3 Hz), 6.73 (d, 2H, J=2.3 Hz), 6.60 (m, 4H); MS EST− m/z 578 (M−H)⁻.

(2-Bromo-4-sulfamoylphenyl)ureido fluorescein (8): ¹H NMR (DMSO-d₆, 400 MHz) δ 10.48 (s, 1H), 10.21 (s, 2H), 9.86 (s, 2H), 8.35 (dd, 2H, J=15.8 Hz, J=1.8 Hz), 7.63 (d, 1H, J=8.3 Hz), 7.52 (s, 2H), 7.23 (d, 1H, J=8.3 Hz), 6.71 (d, 2H, J=2.3 Hz), 6.60 (m, 4H); MS ESI⁻ m/z 623 (M−H)⁻.

(2-Iodo-4-sulfamoylphenyl)ureido fluorescein (9): ¹H NMR (DMSO-d₆, 400 MHz) δ 10.45 (s, 1H), 10.19 (s, 2H), 9.78 (s, 2H), 8.35 (dd, 2H, J=15.8 Hz, J=1.8 Hz), 7.68 (d, 1H, J=8.3 Hz), 7.54 (s, 2H), 7.32 (d, 1H, J=8.3 Hz), 6.77 (d, 2H, J=2.31 Hz), 6.62 (m, 4H); MS ESI⁻ m/z 670 (M−H)⁻.

[4-(4-Sulfamoyl-benzylsulfamoyl)-phenyl]-ureido fluorescein (10): ¹H NMR (DMSO-d₆, 400 MHz) δ 10.47 (2s, 2H), 10.21 (s, 2H), 8.28 (d, 2H, J=6 Hz), 7.82 (m, 7H), 7.54 (d, 2H, J=8.2 Hz), 7.36 (s, 2H), 7.25 (d, 1H, J=8.2 Hz), 6.71 (d, 2H, J=1.7 Hz), 6.63 (m, 4H), 4.12 (d, 2H, J=5.9 Hz); MS ESI⁺ m/z 731 (M+H)⁺, 753 M+Na)⁺. ESI⁻ m/z 713 (M−H)⁻.

[4-(5-Sulfamoyl-[1,3,4]thiadiazol-2-ylsulfamoyl)-phenyl]-ureido fluorescein (11): ¹H NMR (DMSO-d₆, 400 MHz) δ 10.44 (s, 2H), 10.19 (s, 3H), 8.46 (s, 1H), 8.40 (m, 1H), 8.21 (dd, 1H, J=9.8 Hz, J=1.8 Hz), 7.86 (m, 3H), 7.70 (m, 2H), 7.24 (dd, 1H, J=12.4 Hz, J=8.3 Hz), 6.71 (d, 2H, J=2.5 Hz), 6.64 (m, 4H); MS. EST− m/z 707 (M−H)⁻.

### Biological Tests

### Penetrability Through Red Cell Membranes

An amount of 10 mL of freshly isolated human red cells thoroughly washed several times with Tris buffer (pH 7.40, 5 mM) and centrifuged for 10 min were treated with 25 mL of a 3 mM solution of sulfonamide inhibitor. Incubation has been done at 37° C. with gentle stirring, for periods of 30-120 min. After the incubation times of 30 min, 60 min and 48 hours, respectively, the red cells were centrifuged again for 10 min, the supernatant discarded, and the cells washed three times with 10 mL of the above mentioned buffer, in order to eliminate all unbound inhibitor. The cells were then lysed in 25 mL of distilled water, centrifuged for eliminating membranes and other insoluble impurities. The obtained solution was heated at 100° C. for 5 minutes (in order to denature CA-s) and sulfonamides possibly present have been assayed in each sample by two methods: a HPLC method (Gomaa, Z. S.; Biomed. Chromatogr. 1993, 7, 134-135); and spectrophotometrically (Abdine, H.; et al.; J. Assoc. Off. Anal. Chem. 1978, 61, 695-701).

HPLC: A variant of the above methods of Gomaa has been developed by us, as follows: a commercially available 5 μm Bondapak C-18 column was used for the separation, with a mobile phase made of acetonitrile—methanol—phosphate buffer (pH 7.4) 10:2:88 (v/v/v), at a flow rate of 3 mL/min, with 0.3 mg/mL sulphadiazine (Sigma) as internal standard. The retention times were: 12.69 min for acetazolamide; 4.55 min for sulphadiazine; 10.54 min for benzolamide; 12.32 min for aminobenzolamide; 8.76 min for 7; 4.12 min for 8; 6.50 min for 5b; and 6.27 min for 5c. The eluent was monitored continuously for absorbance (at 254 nm for acetazolamide, and wavelength in the range of 270-310 nm in the case of the other sulfonamides).

Spectrophotometrically: A variant of the pH-induced spectrophotometric assay of Abdine et al. (Abdine, H.; et al.; J. Assoc. Off. Anal. Chem. 1978, 61, 695-701) has been used, working for instance at 260 and 292 nm, respectively, for acetazolamide; at 225 and 265 nm, respectively, for sulfanilamide, etc. Standardized solutions of each inhibitor have been prepared in the same buffer as the one used for the membrane penetrability experiments.

Cell cultures. MDCK and HeLa cells as well as their transfected derivatives were grown in DMEM with 10% FCS (BioWhittaker, Verviers, Belgium) buffered with 22.4 mM bicarbonate and containing supplements as described before (Svastova, E.; et al.; Exp. Cell. Res. 2003, 290, 332-345) To maintain standard experimental conditions, the cells were always plated in 3 ml of culture medium at a density of 0.8−1×10⁶ per 6 cm dish 24 h before the transfer to hypoxia (2% O₂ and 5% CO₂ balanced with N₂) generated in a Napco 7000 incubator, where they were grown for additional 48 h (if not stated otherwise). Parallel normoxic dishes were incubated in air with 5% CO₂. At the end of each experiment, pH of the culture medium was immediately measured using portable ARGUS pH meter with IFSET Hot-Line CupFET pH sensor (Sentron, Roden, Netherlands), then the medium was harvested for determination of lactic acid content with standard assay kit (Sigma, St. Louis, Mo.), the cells were counted to ensure that the resulting cultures are comparable and parallel dishes were processed either for immunofluorescence or extracted for immunoprecipitation and/or immunoblotting.

Sulfonamide treatment of cells. The sulfonamides were dissolved in PBS with 20% DMSO at 100 mM concentration and diluted in a culture medium to a required final concentration just before their addition to cells. Immediately after beginning of the treatment with sulfonamides, the cells were transferred to hypoxia and incubated for 48 h. Parallel cultures were maintained for the same time period in normoxia. At the end of the experiment, pH of the culture medium was measured as described above and the binding of the fluorescent sulfonamide 5c to living cells, which were washed three times with PBS, was viewed by a Nikon E400 epifluorescence microscope equipped with PlanFluor objectives 20× and photographed. Images were acquired by Nikon Coolpix 990.

Cloning of CA IX mutants and transfection. Cloning of the deletion mutants of CA IX that lack either the N-terminal PG domain or the central CA domain was performed as described (Zat'ovicova, M.; et al.; J. Immunol. Methods 2003, 282, 117-134). MDCK and HeLa cell lines constitutively expressing CA IX protein or its mutated forms were obtained by cotransfection of individual recombinant plasmids pSG5C-CA IX, pSG5C-ΔCA and pSG5C-ΔPG with pSV2neo plasmid in a 10:1 ratio using a GenePorter II transfection kit from Gene Therapy Systems (San Diego, Calif.). The transfected cells were subjected to selection in the presence of 500-1000 μg/ml G418 (Life Technologies, Gaithersburg, Md.), cloned, tested for expression of CA IX and expanded. At least three clonal cell lines expressing each CA IX form were analyzed to eliminate the effect of clonal variations. The cells cotransfected with empty pSG5C and pSV2 neo and subjected to the same selection and cloning procedures were used as negative controls.

Indirect immunofluorescence. Cells grown on glass coverslips were fixed in ice-cold methanol at −20° C. for 5 min. Non-specific binding was blocked by incubation with PBS containing 1,% BSA (PBS-BSA) for 30 min at 37° C. The cells were incubated with the hybridoma medium containing CA IX-specific monoclonal antibodies M75 directed to PG domain (Zat'ovicova, M.; et al.; J. Immunol. Methods 2003, 282, 117-134) or V/10 directed to CA domain (Zat'ovicova, M.; et al.; J. Immunol. Methods 2003, 282, 117-134) for 1 h at 37° C., washed four times with PBS-BSA, incubated with FITC-conjugated anti-mouse IgG (Vector Laboratories, Burlingame, Calif.) and washed as before. Finally, the cells were mounted onto slides in mounting medium with Citifluor (Agar Scientific, Essex, UK), viewed by Nikon E400 microscope and photographed.

Immunoblotting. Cell monolayers were rinsed twice with cold PBS and solubilised in ice-cold RIPA buffer (1% Triton X-100 and 1% deoxycholate in PBS) containing COMPLETE cocktail of protease inhibitors (Roche Diagnostics GmbH, Mannheim, Germany) for 30 min on ice. The extracts were collected, cleared by centrifugation at 15 000 rpm for 10 min at 4° C. and stored at −80° C. Protein concentrations of extracts were quantified using the BCA protein assay reagent (Pierce, Rockford, Ill.). Total cellular extracts (50 μg of proteins/lane) were resolved in 10% SDS-PAGE gel under reducing and non-reducing conditions, respectively. The proteins were then transferred to PVDF (polyvinylidene difluoride) membrane (Amersham Pharmacia Biotech, Little Chalfont Buckinghamshire, UK). After blocking in 5% non-fat dry milk with 0.2% Nonidet P40 in PBS, the membrane was probed with MAbs (undiluted hybridoma medium), washed and treated with secondary anti-mouse HRP-conjugated swine antibody diluted 1/7500 (Sevapharma, Prague, Czech Republic). The protein bands were visualized by enhanced chemiluminiscence using the ECL kit (Amersham Pharmacia Biotech, Little Chalfont Buckinghamshire, UK).

Cell biotinylation and immunoprecipitation, Cells were washed with ice-cold buffer A (20 mM sodium hydrogen carbonate, 0.15 M NaCl, pH 8.0) and incubated for 60 nm in at 4° C. with buffer A containing 1 mg of NHS-LC-Biotin (Pierce, Rockford, Ill.). After biotinylation, the cells were washed 5 times with buffer A and solubilized in RIPA as described above. Monoclonal antibody V/10 in 1 ml of hybridoma medium was bound to 25 μl 50% suspension of Protein-A Sepharose (Pharmacia, Uppsala, Sweden) for 2 h at RT. Biotinylated cell extract (200 μl) was pre-cleared with 20 μl of 50% suspension of Protein-A Sepharose and then added to the bound MAb. Immunocomplexes collected on Protein-A Sepharose were washed, boiled 5 min in Laemmli loading buffer with or without 2-mercaptoethanol and separated by SDS-PAGE gel (10%) electrophoresis. Afterwards, the proteins were transferred to a PVDF membrane and revealed with peroxidase-conjugated streptavidin (1/1000, Pierce, Rockford, Ill.) followed by enhanced chemiluminiscence.

### CA Inhibition.

Inhibition data against isozymes I, II and IX with some preferred compounds 5a-5k shown in Scheme 3 are reported Table 1. (Khalifah, R. G.; J. Biol. Chem. 1971, 246, 2561-2573)

Data of some standard inhibitors, shown in the following Scheme 2, as well as compounds previously reported by our group are also shown for comparison.

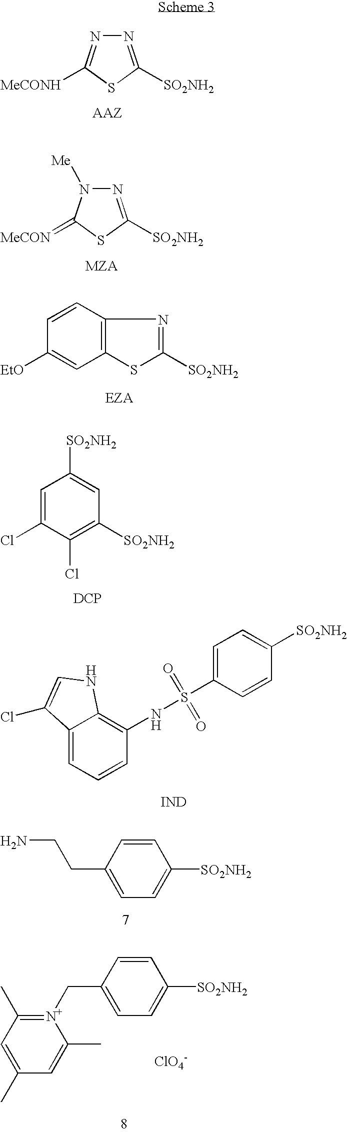

TABLE 1
Inhibition data of fluorescent sulfonamides 5 reported in the present
paper and standard CA inhibitors, against isozymes I, II and IX.

| | K_{I}* (nM) | | |
|---|---|---|---|
| Inhibitor | hCA I^{a} | hCA II^{a} | hCA IX^{b} |
| AZA | 900 | 12 | 25 |
| MZA | 780 | 14 | 27 |
| EZA | 25 | 8 | 34 |
| DCP | 1200 | 38 | 50 |
| IND | 31 | 15 | 24 |
| 5a | 1500 | 41 | 29 |
| 5b | 1450 | 44 | 26 |
| 5c | 1300 | 45 | 24 |
| 5d | 1200 | 40 | 25 |
| 5e | 980 | 47 | 30 |
| 5f | 950 | 52 | 32 |
| 5g | 1100 | 43 | 35 |
| 5h | 1070 | 40 | 31 |
| 5i | 1400 | 52 | 34 |
| 5j | 630 | 34 | 20 |
| 5k | 480 | 27 | 16 |
| 7 | 2100 | 160 | 33 |
| 8 | 7000 | 50 | 38 |

*Errors in the range of 5-10% of the reported value (from 3 different assays).
^{a}Human (cloned) isozymes, by the CO₂ hydration method;
^{b}Catalytic domain of human, cloned isozyme, by the CO₂ hydration method.

Data of the 4-aminoethylbenzenesulfonamide 7 (Vullo, D.; et al.; Bioorg. Med. Chem. Lett. 2003, 13, 7005-1009) and the 2,4,6-trimethylpyridinium derivative of homosulfanilamide 8, (Scozzafava, A.; et al.; J. Med. Chem. 43, 292-300 (2000); Pastorekova, S.; et al.; Bioorg. Med. Chem. Let. 2004, 14, 869-873) used in the ex vivo studies are also shown.

The compounds 1-11 have been tested as in vitro inhibitors of the carbonic anhydrase isozymes I, II (cytosolic forms) and IX and XII (tumor-associated isoform, with transmembrane localization) (Table 2). It may be observed that these compounds are excellent inhibitors of the tumor associated isozymes IX and XII (K_{I}-s in the range of 6-46 nM against CA IX, and 3-18 nM against CA XII, respectively), being at the same time less effective inhibitors of the ubiquitous cytosolic isozymes I and II (K_{I}-s in the range of 410-1900 nM against isoform CA I, and 13-76 nM against CA II).

TABLE 2
Inhibition data of the new fluorescent sulfonamides 1-11
reported here against isozymes I, II, IX and XII

K_{I}* (nM)

| Inhibitor | hCA I^{a} | hCA II^{a} | hCA IX^{b} | hCA XII^{b} |
|---|---|---|---|---|
| 1 | 1700 | 36 | 21 | 11 |
| 2 | 1800 | 43 | 30 | 9 |
| 3 | 1900 | 76 | 46 | 18 |
| 4 | 1560 | 40 | 21 | 8 |
| 5 | 1340 | 35 | 18 | 7 |
| 6 | 1200 | 40 | 21 | 10 |
| 7 | 1330 | 43 | 22 | 12 |
| 8 | 1300 | 41 | 20 | 9 |
| 9 | 1350 | 43 | 21 | 7 |
| 10 | 520 | 14 | 7 | 5 |
| 11 | 410 | 13 | 6 | 3 |

*Errors in the range of 5-10% of the reported value (from 3 different assays).
^{a}Human (cloned) isozymes, by the CO₂ hydration method;
^{b}Catalytic domain of human, cloned isozyme, by the CO₂ hydration method.

### Ex Vivo Penetration Through Red Blood Cell Membranes

Levels of sulfonamides in red blood cells after incubation of human erythrocytes with millimolar solutions of inhibitor for various periods of time (starting with 30-60 min till 48 hours) are shown in Table 3. The methods are disclosed in Gomaa, Z. S.; Biomed. Chromatogr. 1993, 7, 134-135; Abdine, H.; et al.; J. Assoc. Off. Anal. Chem. 1978, 61, 695-701 and Wistrand, P. J.; Lindqvist, A. in Carbonic Anhydrase—From Biochemistry and Genetics to Physiology and Clinical Medicine, Botrè, F.; Gros, G.; Storey, B. T. Eds., VCH, Weinheim, 1991, pp. 352-378.

TABLE 3
Levels of sulfonamide CA inhibitors (μM) in red blood
cells at 30 and 60 min, after exposure of 10 mL of blood
to solutions of sulfonamide (3 mM sulfonamide in 5 mM Tris
buffer, pH 7.4). The concentrations of sulfonamide has
been determined by two methods: HPLC; and electronic spectroscopy
(ES) - see Experimental for details.

| | [sulfonamide], μM* | | | | | |
|---|---|---|---|---|---|---|
| | t = 30 min | | t = 60 min | | t = 48 h | |
| Inhibitor | HPLC^{a} | ES^{b} | HPLC^{a} | ES^{b} | HPLC^{a} | ES^{b} |
| AZA | 136 | 139 | 160 | 167 | 163 | 168 |
| MZA | 170 | 169 | 168 | 168 | 167 | 169 |
| 7 | 132 | 138 | 162 | 165 | 167 | 168 |
| 8 | 0.3 | 0.5 | 0.4 | 0.5 | 0.3 | 0.5 |
| 5b | 0.5 | 0.8 | 0.8 | 0.8 | 10.1 | 2.5 |
| 5c | 0.4 | 0.9 | 0.6 | 1.2 | 10.4 | 3.0 |

*Standard error (from 3 determinations) < 5% by:
^{a}the HPLC method¹⁸;
^{b}the electronic spectroscopic method (Abdine, H.; et al.; J. Assoc. Off. Anal. Chem. 1978, 61, 695-701).

### CA IX-Mediated Acidification of the Extracellular pH in Hypoxia and its Inhibition by Sulfonamides

The CA EX-transfected MDCK cells and mock-transfected controls used for determining the pHe values in hypoxia (H, 2% O₂)/normoxia (N, 21% O₂) were analyzed by immunoblotting using the CA IX monoclonal anti-body (Mab) M75, (Zat'ovicova, M.; et al.; J. Immunol. Methods 2003, 282, 117-134). Transfected MDCK cells were analysed by immunofluorescence and the values of pHe and lactate concentrations in the cells grown in the constant medium volumes were determined. Five independent experiments with three different clones of the transfectants and three parallel dishes for each clone were performed. Results are illustrated on histogram showing the mean values and standard deviations.

The values of pHe and lactate concentrations in the cells grown in the constant medium volumes is shown in FIG. 1.

The binding of three different sulfonamide CAIs (including the fluorescent derivative 5c according to the invention) to hypoxic MDCK-CA IX cells and their effect on the pHe are shown in FIG. 2.

The sulfonamides 8, 7 and 5c (in concentrations of 0.1 mM and 1 mM) respectively were added to MDCK-CA IX cells just before their transfer to hypoxia and pHe was measured 48 h later. At least three independent experiments with three parallel dishes per sample were performed for each inhibitor.

Treatment of the tumor HeLa and SiHa cervical carcinoma cells with the fluorescent sulfonamide 5c and its effect on the tumor pH are shown in FIG. 3.

HeLa and SiHa cervical carcinoma cells were incubated for 48 h in normoxia and hypoxia, respectively, either in the absence or in the presence of 1 mM 5c. Mean differences in the pH values determined in the treated versus control dishes are shown on the histogram with indicated standard deviations. The experiment was repeated three times using at least three parallel dishes for each sample.

Data of Table 1 show the inhibition properties against the cytosolic isozymes hCA I and II, as well as the transmembrane, tumor-associated isozyme hCA IX of the compounds of the present invention, as well as standard, clinically used inhibitors (acetazolamide AAZ, methazolamide MZA, ethoxzolamide EZA, dichlorophenamide DCP and indisulam IND) or some other sulfonamides previously investigated by us for targeting the tumor-associated Cas (such as 7 and 8) (Bioorg. Med. Chem. Lett. 2003, 13, 1005-1009; Scozzafava, A.; et al.; J. Med. Chem. 43, 292-300 (2000); Pastorekova, S.; et al.; Bioorg. Med. Chem. Let. 2004, 14, 869-873). The following should be noted regarding data of Table 1: (i) the fluorescent sulfonamides 5 reported here behave as moderate—weak inhibitors against the slow cytosolic isozyme hCA I, with inhibition constants in the range of 480-1500 Nm. It is in fact well-known (Carbonic anhydrase—its inhibitors and activators, CRC Press (Taylor and Francis Group), Boca Raton, Fla., 2004, pp. 1-363, and references cited therein) that this isozyme has a lower affinity for sulfonamides, as compared to hCA II or hCA IX. Thus, these fluorescent sulfonamides show similar affinities for this isozyme as the clinically used compounds AZA, MZA or DCP, whereas ethoxzolamide EZA and indisulam IND are much more potent CA I inhibitors (K_{I}-s in the range of 25-31 Nm). Compounds 7 and 8 also show modest hCA I inhibitory properties (Table 1); (ii) against the major cytosolic isozyme hCA II; the fluorescent sulfonamides 5 show a very compact behaviour as efficient inhibitors, with K_{I}-s in the range of 27-52 Nm.

In fact, several recent X-ray crystallographic studies on adducts of hCA II with sulfonamides showed that the tails attached to the aromatic/heterocyclic sulfonamide scaffold make extensive contacts with amino acid residues both in the middle as well as at the entrance of the active site, leading thus to nanomolar affinity for the enzyme (Bioorg. Med. Chem. Lett. 2004, 14, 217-223; J. Med. Chem. 2004, 47, 550-557; J. Enz. Inhib. Med. Chem. 2003, 18, 303-308; Bioorg. Med. Chem. Lett. 2003, 13, 2759-2763; Bioorg. Med. Chem. Lett. 2004, 14, 2357-2361). Thus, the best hCA II inhibitors in this series of sulfonamides were the aminobenzolamide derivative 5k and the sulfanilyl-homosulfanilamide 5j, but the other compounds—as mentioned above—were only slightly less inhibitory than 5j,k. These compounds are less efficient CA II inhibitors as compared to the clinically used derivatives, which typically showed K_{I} values I the range of 8-15 Nm (DCP is the less effective such inhibitor, with a K_{I} of 38 Nm). The simple derivatives 7 and 8 are also less effective CA II inhibitors (K_{I}-s in the range of 50-160 Nm); (iii) against the tumor-associated isozyme CA IX, the fluorescent sulfonamides 5 showed very good inhibitory properties, with K_{I}-s in the range of 16-35 Nm. Similarly to the situation observed for CA II, there are not important variations of activity for the diverse structures included in the study, and the explanation may be the one mentioned above. But it is important to note that all these compounds act as better Hca IX than Hca II inhibitors, which constitutes a remarkable finding, since a possible drugs based on CA IX inhibitors should bind as much as possible to the target, cancer-associated isozymes (i.e., CA IX and XII) but not to the other ubiquitous CA isozymes, such as CA II, IV or V. Probably this is due to the fact that the Hca IX active site is larger than that of the cytosolic isozyme Hca II, as already reported earlier by us (Bioorg. Med. Chem. Let. 2004, 14, 869-873). It must also be noted that the CA IX inhibitory properties of these new sulfonamides 5 are in the same range as those of the clinically used sulfonamides, including indisulam, an antitumor sulfonamide in clinical trials.

### Ex Vivo Penetration Through Red Blood Cell Membranes

Levels of sulfonamides 5b,c, 7, 8, AZA and MZA in red blood cells (which contain high concentrations of isozymes I and II, i.e., 150 μM Hca 1 and 20 μM Hca II, but not the membrane-bound CA IV or CA IX; Carbonic Anhydrase—From Biochemistry and Genetics to Physiology and Clinical Medicine, Botrè, F.; Gros, G.; Storey, B. T. Eds., VCH, Weinheim, 1991, pp. 352-378) after incubation periods of 30 min, 60 min or 48 hours were determined in order to investigate the penetrability of these compounds through biological membranes. Since Hca IX is a transmembrane protein with the active site exposed out of the cell, membrane-impermeant derivatives (or derivatives with decreased permeability) may lead to the selective inhibition of Hca IX and not of the cytosolic CA isozymes CA I or II. This is considered a very desirable property of a future drug belonging to this class of compounds. We have already shown previously that the positively-charged, pyridinium-substituted sulfonamides of which 8 is a representative, are indeed membrane-impermeable, in contrast to classical sulfonamides which cross membranes easily due to the fact they are non polar and uncharged (although in equilibrium with the ionised sulfonamide, which is the species binding to the enzyme active site) (Scozzafava, A.; et al.; J. Med. Chem. 43, 292-300 (2000); Pastorekova, S.; et al.; Bioorg. Med. Chem. Let. 2004, 14, 869-873; Supuran, C. T.; et al.; J. Enz. Inhib. Med. Chem. 2004, 19, 269-273.

Indeed, it may be observed (Table 3) that the uncharged sulfonamides AZA, MZA and aminoethylbenzenesulfonamide 7, easily penetrate through biological membranes, practically saturating red blood cells (RBCs) after 1 hour. After 48 hours, identical levels (within the limits of experimental errors) of these three sulfonamides in RBCs were observed. On the contrary, the pyridinium, charged compound 8, has been detected only in very small amounts within the RBCs, proving that it is unable to penetrate through the membranes, obviously due to its cationic nature. Even after incubation times as long as 48 hours only traces of the cationic sulfonamide were present inside the RBCs, as proved by the two assay methods used for their identification in the cell lysate, which were in good agreement with each other (the very small amount of sulfonamide detected may be due to contamination of the lysates with minute amount of membranes) (Table 3). The fluorescein sulfonamide derivatives 5b and 5c investigated here showed a decreased membrane permeability at exposure times of 30-60 min, but were slightly more permeant after 48 hours of exposure. These findings may be explained by the fact that due to the presence of the carboxylic acid moiety in these compounds, and in the conditions of our experiments (pH 7.4), most of the fluorescent sulfonamide is in anionic, carboxylate form, which leads to a decreased penetration through membranes, similarly to the cationic sulfonamide 8. Still, these carboxylates are in chemical equilibrium with the corresponding acids—neutral molecules—which are membrane-permeant, and this may explain why after 48 hours of incubation, some sulfonamide crossed the membranes (on the other hand 8 is not in equilibrium with any neutral molecule and this is the reason why the compound cannot cross membranes even after 48 hours of incubation with RBCs). Still, these levels are quite small, and considering the fact that compounds 5 showed a better affinity for hCA IX than for hCA II, in vivo we hypothesize that the cancer-associated, transmembrane isozyme IX is predominantly inhibited by these compounds.

### CA IX-Mediated Acidification of the Extracellular pH in Hypoxia and its Inhibition by Sulfonamides

Expression of CA IX in tumor cells is strongly induced by hypoxia simultaneously with various components of anaerobic metabolism and acid extrusion pathways (Mol. Med. Today 2000, 6, 15-19; Clin. Cancer Res. 2002, 8, 1284-1291). This could complicate a discrimination of CA IX contribution to resulting overall change in pHe. Therefore, we used as a model MDCK immortalised canine kidney epithelial cells that do not contain own CA IX, but were stably transfected to express human CA IX protein in a constitutive manner. As shown by immunoblotting analysis, levels of CA IX in MDCK-CA IX transfectants were comparable between the hypoxic cells maintained for 48 h in 2% O₂ and the normoxic cells incubated in 21% O₂ (FIG. 1a). In immunofluorescence analysis, CA IX was predominantly localized at the surface of both normoxic and hypoxic cells (FIG. 1b), although the membrane staining in hypoxic cells was less pronounced due to hypoxia-induced perturbation of intercellular contacts as described in (Svastova, E.; et al.; Exp. Cell. Res. 2003, 290, 332-345). Measurement of the culture medium pH revealed that the hypoxic incubation led to expected extracellular acidification in CA IX-positive as well as CA IX-negative cell cultures when compared to their normoxic counterparts (FIG. 1c). However, upon the mutual comparison of the hypoxic cells it became evident that pHe was significantly decreased in cells containing CA IX. A minor difference between the pHe values of CA IX-negative versus CA IX-positive cells was found in normoxia. Taking into account a steady, hypoxia-independent level of CA IX in MDCK-CA IX cells, this finding indicated that hypoxia activated the catalytic performance of CA IX which resulted in enhanced pHe acidification.

To exclude the possibility that hypoxia-induced acidification was caused by increased production of lactic acid, we measured pHe and determined corresponding lactate concentrations in media from both CA IX-negative and CA IX-positive transfectants (FIG. 1d). The cells maintained in hypoxia for 16 h displayed no significant differences in pHe values when compared to parallel normoxic cultures. In both conditions, culture media of CA IX-transfected cells had slightly lower pH values than the media from the control mock-transfected cells (FIG. 1d). After 48 h, pHe of the normoxic cells decreased irrespective of whether they contained CA IX or not. This pHe decrease was apparently coupled with the accumulation of lactate, whose final concentration was similar in CA IX-positive and CA IX-negative cells. Hypoxic treatment of MDCK-mock cultures for 48 h resulted in small pHe decrease compared to the parallel normoxic cells, whereas the medium of MDCK-CA IX cells was considerably more acidic then its normoxic counterpart. The small pHe decline noted in the hypoxic mock-transfected cells could be assigned to increased concentration of lactic acid generated consequently to hypoxia-induced metabolic changes. It could be also responsible for the corresponding proportion of medium acidification in CA IX-expressing cells. However, because there was practically no difference between the lactate production in 48 h cultures of CA IX-positive and CA IX-negative cells, the remaining pHe decrease could be explained by the catalytic activity of CA IX.

If the enzymatic activity of CA IX was responsible for the augmented acidification, then it could be blocked by sulfonamides, which efficiently inhibit carbonic anhydrases by a well-understood mechanism (Carbonic anhydrase—its inhibitors and activators, CRC Press (Taylor and Francis Group), Boca Raton, Fla., 2004, pp. 1-363, and references cited therein). Moreover, the fluorescent sulfonamide 5c was used for the treatment and fluorescence analysis of both CA IX-positive and CA IX-negative cells incubated either in normoxia or in hypoxia for 48 h. In a perfect agreement with the previous data, the fluorescence signal produced by 5c was detected only in the hypoxic MDCK-CA IX cells, but was absent from their normoxic counterparts and from both hypoxic and normoxic mock-transfected controls. This observation indicates that 5c did not interact with other CA isoforms and that it binds only to hypoxia-activated CA IX. Altogether, these results offer a reliable proof that CA IX activity is essential for the medium acidification in hypoxic MDCK-CA IX cells, and that this acidification is reversed by inhibiting CA IX with sulfonamides.

To see, whether the phenomenon of CA IX-mediated acidification is of any significance in tumor cells expressing endogenous CA IX, we examined the effect of sulfonamide 5c on the pHe of cervical carcinoma cells HeLa and SiHa, respectively. Under hypoxia, tumor cells co-ordinately express elevated levels of multiple HIF-1 targets, including CA IX (Semenza, G. L. Nature Rev. Cancer 2003, 3, 721-732). In addition, activity of many components of the hypoxic pathway and related pH control mechanisms, such as ion transport across the plasma membrane, are abnormally increased in order to maintain neutral intracellular pH (Mol. Med. Today 2000, 6, 15-19; Clin. Cancer Res. 2002, 8, 1284-1291). This explains considerably decreased pHe of hypoxic versus normoxic HeLa and SiHa cells (FIG. 3). The acidosis was reduced by 5c, in support of the idea that activation of CA IX is just one of many consequence of hypoxia. Moreover, 5c binds to hypoxic HeLa and SiHa cells that express elevated levels of CA IX, but not to normoxic cells with diminished CA IX expression. As indicated by the ability to bind this fluorescent inhibitor, CA IX expressed in the hypoxic tumor cells was catalytically active. Noteworthy, exclusive binding of the fluorescent inhibitor to hypoxic cells with activated CA IX offers an attractive possibility for the use of similar sulfonamide-based compounds for imaging purposes, e.g. to visualize the hypoxic tumors in positron emission tomography. In addition, CA IX-selective sulfonamide derivatives may potentially serve as components of therapeutic strategies designed to decrease pHe in tumor microenvironment and thereby reduce tumor aggressiveness and drug uptake (Mol. Med. Today 2000, 6, 15-19; Clin. Cancer Res. 2002, 8, 1284-1291; Teicher, B. A.; et al.; Anticancer Res. 1993, 13, 1549-1556).

1. Compounds of formula (I) A-(Q)ₙ-Ar—SO₂NHR wherein A is the moiety of a fluorescent dye;
Q is the group —NH—CX—NH—(R₁)H₁ or —NH—CX—NH—NH—(R₁)ₘ, wherein X is O or S, R₁ is a C₁-C₄ alkylene, m is the number 0 or 1; n is the number 0 or 1; Ar is a Ce—C₁O aromatic or a heteroaromatic group containing at least one heteroatom selected from the group consisting of oxygen, nitrogen and sulphur, said aromatic and heteroaromatic groups optionally being substituted by at least one, halogen atom;
R is hydrogen or a B—SO₂NH₂ group, wherein B is a (C₁-C₄)ᵣ alkylene-aromatic or (C₁-C₄)ᵣ alkylene-heteroaromatic group, wherein r is 0 or 1; with the exclusion of the (4-sulfamoylphenylmethyl)thioureido fluorescein, their pharmaceutically acceptable hydrates, solvates and salts.

2. A compound according to claim 1, wherein Ar is phenyl, optionally substituted by at least one halogen atom and R is H.

3. A compound according to claim 2, wherein Q is the group —NH—CX—NH—(R₁)Hi, wherein X is S, m is 0, Ar is phenyl, optionally substituted by at least one halogen and R is H.

4. A compound according to claim 1, wherein R is a B—SO₂NH₂ group, wherein B is an aromatic or heteroaromatic group.

5. A compound according to claim 4, wherein Q is the group —NH—CX—NH—(R₁)Hi, wherein X is S, m is 0, Ar is phenyl and R is B—SO₂NH₂ group, wherein B is 1,3,4-thiadiazol-2-yl.

6. A compound according to claim 1, wherein A is a fluorescein residue.

7. A compound according to claim 1, selected from the group consisting of: (4-Sulfamoylphenyl)thioureido fluorescein;
(4-Sulfamoylphenylethyl)thioureido fluorescein; (2-Iodo-4-sulfamoylphenyl)thioureido fluorescein; (3-Sulfamoylphenyl)thioureido fluorescein; [4-(4-Sulfamoyl-benzylsulfamoyl)-phenyl]-thioureido fluorescein; [4-(5-Sulfamoyl-[1,3,4]thiadiazol-2-ylsulfamoyl)-phenyl]-thioureido fluorescein.

8. A compound according to claim 1, selected from the group consisting of: (4-Sulfamoylphenyl)ureido fluorescein;
(3-Sulfamoylphenyl)ureido fluorescein;
(2-Sulfamoylphenyl)ureido fluorescein;
(4-Sulfamoylphenylmethyl)ureido fluorescein;
(4-Sulfamoylphenylethyl)ureido fluorescein; (2-Fluoro-4-sulfamoylphenyl)ureido fluorescein;
(2-Chloro-4-sulfamoylphenyl)ureido fluorescein;
(2-Bromo-4-sulfamoylphenyl)ureido fluorescein;
(2-Iodo-4-sulfamoylphenyl)ureido fluorescein;
[4-(4-Sulfamoyl-benzylsulfamoyl)-phenyl]-ureido fluorescein; [4-(5-Sulfamoyl-[1,3,4]thiadiazol-2-ylsulfamoyl)-phenyl]-ureido fluorescein.

9. A process for the preparation of the compounds of claim 1, comprising the reaction of a compound of formula (II) A-NH2, with a compound of formula (III) XCN—(R₁)ₘ—Ar—Sθ2NHR.

10. A process for the preparation of the compounds of claim 1, comprising the reaction of a compound of formula (IV) A-NCX, with a compound of formula (V) H2N—(R₁)ₘ—Ar—SO2NHR.

11. One of the compounds of claim 1 as a probe for the identification of hypoxic tumors.

12. The compound according to claim 11, in which said tumor is Carbonic Anhydrase IX-positive.

13. The compound according to claim 11, in which said identification is carried out by positron-emission tomography.

14. One of the compounds of claim 1 for the preparation of a reagent for the detection of Carbonic Anhydrase in a living subject.

15. The compound according to claim 14, wherein said subject is human.

16. The compound according to claim 14, wherein said Carbonic Anhydrase is Carbonic Anhydrase IX.

17. The compound according to claim 14, in which said detection is carried out by positron-emission tomography.

18. One of the compounds of claim 1 for the reparation of a medicament.

19. One of the compounds of claim 1 for the preparation of a medicament having carbonic anhydrase inhibiting action.

20. The compound according to claim 19, wherein said medicament has a selective inhibiting activity towards carbonic anhydrase isozyme IX.

21. The compound according to claim 19, in which said medicament is effective for the treatment of a hypoxic tumor.

22. The compound according to claim 19, in which said medicament is effective for reversing acidification of a hypoxic tumor.

23. The compound according to claim 19, in which said medicament is effective for treating a Carbonic Anhydrase IX-positive tumor.

24. The compound according to claim 19, wherein said tumor is selected from the group consisting of kidney, breast, lung, head and neck, gliomas, mesothelomas, stomach, colon, biliary, pancreatic, cervix, endometrial, squamal/basal cell carcinomas.

25. The compound according to claim 19, in which said medicament is used in combination therapy.

26. The compound according to claim 25, wherein said therapy is antitumor therapy.

27. A pharmaceutical composition comprising a compound of claim 1 in admixture with at least one pharmaceutically acceptable ingredient.

28. A fluorescent reagent comprising a compound of claim 1.

29. A diagnostic kit comprising a compound of claim 1.

30. A composition for tumor imaging comprising a compound of claim 1.
